(19) United States
(12) Patent Application Publication
(10) Pub. No.: US 2023/0015152 A1
(43) Pub. Date: Jan. 19, 2023

(54) MOUTHPIECE

(71) Applicant: Mi Ra PARK, Seoul (KR)

(72) Inventor: Mi Ra PARK, Seoul (KR)

(21) Appl. No.: 17/641,027

(22) PCT Filed: Sep. 9, 2019
(86) PCT No.: PCT/KR2019/011628

§ 371 Date: Sep. 6, 2022

(51) Int. Cl. A61C 7/08; A61N 1/05; A61N 5/06; A61C 7/10

(52) U.S. Cl. CPC A61C 7/08; A61N 1/0548; A61N 5/0624; A61C 7/10; A61N 2005/0606; A61N 2005/0651; A61N 2005/0659

(57) ABSTRACT

Provided is a mouthpiece which is mounted in an oral cavity for use in changing the shape of the skull including the maxillary bone and is composed of a plurality of segment pieces, at least some of the segment pieces being configured to approach or be spaced apart from each other by means of a screw, wherein the surfaces of the segment pieces facing or contacting the skin in the oral cavity are configured such that the segment pieces overlap with each other, such that a gap does not occur in spite of the approaching or spacing-apart movement of the segment pieces relative to each other.

### TECHNICAL FIELD

The present invention relates to a mouthpiece, and more particularly, to a mouthpiece minimizing a risk of inflammation caused by contact with foods and bacteria by preventing a palatine tissue from being exposed through a gap between segment pieces even in case of expansion by a screw of the mouthpiece, minimizing a downward flow of a soft tissue and a hard tissue of a palate by preventing the palatine tissue from being exposed through the gap between the segment pieces even in case of the expansion by the screw of the mouthpiece, improving a treatment effect through pressing of the palate by firmly supporting the palate through an overlap structure of the segment pieces of the mouthpiece, being made of a material having surface roughness (Ra) of 0.2 to 0.4 so that a bacterial film is not formed, maximizing an action force in an oral cavity by canceling a reaction force vector to an action force vector caused by the screw with a support force vector caused by a external head gear, and including a generator for converting a masticatory force in the oral cavity into electrical energy or a power storage for storing the electrical energy in order to drive actuation devices such as a low frequency generator, an infrared or ultraviolet LED, a screw drive motor, and a wireless communication module with an external terminal and sensors such as a screw actuation amount sensor.

### BACKGROUND ART

In general, a maxillofacial correction device used for resolving malocclusion of teeth is widely known. The maxillofacial correction device includes a mouthpiece acting to a maxillar. The mouthpiece includes a plurality of segment pieces, and at least a portion of the segment pieces perform approaching or spacing-apart movement and expand the maxillar by a pressing force generated when spaced apart from each other.

Alternatively, the maxillofacial correction device may include a mouthpiece and a head gear for applying a traction force to the mouthpiece while supporting the mouthpiece. The RAMPA device is an example of the device including the mouthpiece and the head gear.

The patent document below discloses a maxillofacial orthotic device including a maxillary expanding unit mounted to a maxillar in a mouth of a human and a towed unit connected with the maxillary expanding unit and towed in a forward direction of the human. The maxillary expanding unit includes: a left contact part contacting an inner side of left maxillary teeth of the human; a right contact part contacting an inner side of right maxillary teeth of the human; an adjustment part connecting the left contact part and the right contact part while varying a distance between the left contact part and the right contact part; a left hole defined in the left contact part; and a right hole defined in the right contact part. The towed unit includes: a left wire part having one end inserted to the left hole and the other end protruding out of the mouth; a right wire part having one end inserted to the right hole and the other end protruding out of the mouth; a connection tube formed on one of the left wire part and the right wire part and extending in a direction in which the distance between the left contact part and the right contact part is varied by the adjustment part; and a connection wire formed on the other of the left wire part and the right wire part and being slidable in the connection tube.

As illustrated in FIG. 3, the mouthpiece in this technology is configured such that a width between a left upper part 43 and a right upper part 44, which are divided in a left and right direction, is varied by a central expansion screw part 45. The varying width has a maximum of about 11 mm and a practical value of about 8 mm to 9 mm (paragraph <134>). Thus, the maxillar may be expanded in the left and right direction. Also, the mouthpiece in this technology is manufactured by pouring a typical correction resin into a frame and then curing the resin.

### RELATED ART DOCUMENT

[Patent Document]
(Patent document 1) Korean Patent Registration No. 10-0902533

### DISCLOSURE OF THE INVENTION

### Technical Problem

In the above patent document, as the left upper part 43 and the right upper part 44 are spaced apart from each other, a gap therebetween is generated with a maximum of 11 mm A similar configuration is exemplified in FIG. 9a of the present application. In FIG. 9a, a maxillary expansion part of the mouthpiece includes a rearmost reference plate 41 and moving plates 42 and 43 that respectively move in a forward left side and a forward right side relative to the reference plate 41, and a screw 46 is installed therebetween. Also, a holder 47 having a hollow pipe shape, which is used to connect an external wire to the reference plate 41 is provided.

In this configuration, when the screw 46 acts to expand the maxillar, and the moving plates 42 and 43 move, a gap occurs as illustrated in FIG. 9a. As a skin tissue of the palate is pushed out or exposed through this gap, a risk of inflammation caused by contact with foods is increased. An example of an inflammation reaction is exemplified in FIG. 9b. In FIG. 9b, a downward flown soft tissue of the palate is shown in an oval expressed by a dash-dotted line. FIG. 9a and 9b are photographs of real cases. (The downward flown soft tissue is shown in the oval expressed by the dash-dotted line, and portions except the portion inside the oval are gums reddened due to inflammation.)

In case of a mouthpiece of the related art, only the mouthpiece is worn in an oral cavity without a head gear. In this case, when an action force vector (red arrow) is applied to an anterior upper portion of the palate by expansion of the screw 46 as in FIG. 10a, a reaction force vector (blue arrow) acts in a direction opposite thereto. Here, as a posterior contact portion of the device is displaced downward from a mucous membrane in the oral cavity, a posterior portion of the palatine bone is not supported. Accordingly, the posterior portion protrudes downward. Here, as a cervical portion of the posterior portion in contact with a resin phase of the device is used as a center of rotation, a momentum (less in magnitude than a momentum of a support of the anterior portion) by which the palatine bone rotates downward using a working distance that is a distance between the cervical portion of a molar and the palatine bone is generated. Also, since the posterior portion is not supported, the anterior portion may not be expanded in a forward direction. Also, the device is lifted from the tissue to cause deformation. However, when a patient has a good posture so that a tongue contacts the palatine bone, the downward displacement of posterior teeth may be prevented. In this case, correction of the skull is insufficiently performed as in FIG. 10b.

Here, when the screw acts in one direction by one screw, preferred correction may not be performed as in FIG. 10c, and when the screw acts in two directions by two screws, as the resin portion of the device slips along the cervical portion and moves toward an occlusal surface as in FIG. 10d, a reaction between the mouthpiece and the palatine surface may not be adjusted.

For example, when the mouthpiece of the related art is applied to the RAMPA invented by Dr. Mitani and towed by the head gear, the posterior contact portion of the device is displaced downward from the mucous membrane in the oral cavity, and the posterior portion of the palatine bone is not supported. Accordingly, the anterior portion protrudes in a forward and downward direction. When the device is expanded, the cervical portion of the anterior teeth that is a push point is used as a center of rotation, a distance from the point to the skull is a working distance, and a momentum by which the posterior portion of the palatine bone rotates downward is generated. This is a big momentum because of a long arm length. Also, a forward expansion of the anterior portion is insufficient because the posterior portion is not supported. Also, the device is lifted from the tissue to cause deformation. Also, as a mandibulae rotates in a rear downward direction, a face increases in length. In this case, in the skull as in FIG. 11b, as a forward and backward distance from an anterior cervical vertebrae to mandibular anterior teeth greatly decreases, the maxillary anterior teeth are displaced downward (until cervical vertebrae 3^{rd}). The correction may not be properly performed, e.g., a narrowed respiratory tract is observed.

In the technology of the above patent document, it is described in paragraph <183> that the maxillar grows in the forward direction while lifting the maxillary expander 41 upward. To this end, the mouthpiece is required to be fixed to a head unit 12 and towed in the forward direction. However, the above patent document does not exactly disclose a towed amount.

The present invention is provided to solve the above-described problems of the related art. The mouthpiece according to the present invention may minimize the risk of inflammation caused by contact with foods and bacteria by preventing a palatine tissue from being exposed through a gap between segment pieces even in case of expansion by the screw of the mouthpiece.

Also, as the palatine tissue is prevented from being exposed through the gap between the segment pieces even in case of the expansion by the screw of the mouthpiece, the mouthpiece may minimize a downward flow of a soft tissue and a hard tissue of the palate and firmly support the palate by using an overlap structure of the segment pieces of the mouthpiece to improve a treatment effect by pressing the palate.

Also, the mouthpiece may maximize an action force in the oral cavity by canceling a reaction force vector to an action force vector caused by the screw with a support force vector caused by an external head gear.

Also, the mouthpiece may include a generator for converting a masticatory force in the oral cavity into electrical energy or a power storage for storing the electrical energy in order to drive actuation devices such as a low frequency generator, an infrared or ultraviolet LED, a screw drive motor, and a wireless communication module with an external terminal and sensors such as a screw actuation amount sensor.

Also, the mouthpiece may be made of a material having surface roughness (Ra) of 0.2 to 0.4 so that a bacterial film is not formed.

### Technical Solution

A mouthpiece of the present invention to achieve the objects, which is mounted in an oral cavity for use in changing a shape of a skull including a maxillary bone and is composed of a plurality of segment pieces, at least some of the segment pieces being configured to approach or be spaced apart from each other by means of a screw, is characterized in that surfaces of the segment pieces facing or contacting a skin in the oral cavity are configured such that the segment pieces overlap with each other, so that a gap does not occur in spite of the approaching or spacing-apart movement of the segment pieces relative to each other.

Also, a mouthpiece, which is mounted in an oral cavity for use in changing a shape of a skull including a maxillary bone and is composed of a plurality of segment pieces, at least some of the segment pieces being configured to approach or be spaced apart from each other by means of a screw, is characterized in that at least a portion of the segment pieces are connected to a head gear mounted outside an oral cavity, and a reaction force vector to an action force vector caused by the screw is canceled by a support force vector caused by the head gear.

Also, a mouthpiece, which is mounted in an oral cavity for use in changing a shape of a skull including a maxillary bone and is composed of a plurality of segment pieces, at least some of the segment pieces being configured to approach or be spaced apart from each other by means of a screw, is characterized in that at least a portion of the segment pieces are connected to a connecting wire, the connecting wire is connected to a head gear mounted outside an oral cavity, a blind hole or a through-hole is formed in a holder disposed on a bottom surface of each of the segment pieces connected to the connecting wire, and a projection (convex shape) that is detachably coupled to the blind hole or the through-hole is formed on a latch of the connecting wire.

Also, a mouthpiece, which is mounted in an oral cavity for use in changing a shape of a skull including a maxillary bone and is composed of a plurality of segment pieces, at least some of the segment pieces being configured to approach or be spaced apart from each other by means of a screw, is characterized in that at least one of the segment pieces include a generator for converting a masticatory force in an oral cavity into electrical energy by a piezoelectric phenomenon or a power storage for storing the electrical energy.

Here, the converted electrical energy may drive at least one of a low frequency generator, an infrared LED, an ultraviolet LED, a screw drive motor, a wireless communication module with an external terminal, and a screw actuation sensor.

Also, the mouthpiece is characterized by being made of a material having surface roughness (Ra) of 0.2 to 0.4 so that a bacterial film is not formed.

### Advantageous Effects

According to the present invention, the mouthpiece may minimize the risk of inflammation caused by contact with foods and bacteria by preventing the palatine tissue from being exposed through the gap between the segment pieces even in case of expansion by the screw of the mouthpiece.

Also, as the palatine tissue is prevented from being exposed through the gap between the segment pieces even in case of the expansion by the screw of the mouthpiece, the mouthpiece may minimize the downward flow of the soft tissue and the hard tissue of the palate and firmly support the palate by using the overlap structure of the segment pieces of the mouthpiece to improve the treatment effect by pressing the palate.

Also, the mouthpiece may maximize the action force in the oral cavity by canceling the reaction force vector to the action force vector caused by the screw with the support force vector caused by the external head gear.

Also, the mouthpiece may include the generator for converting the masticatory force in the oral cavity into the electrical energy or the power storage for storing the electrical energy in order to drive the actuation devices such as the low frequency generator, the infrared or ultraviolet LED, the screw drive motor, and the wireless communication module with the external terminal and the sensors such as the screw actuation amount sensor.

Also, the mouthpiece may be made of the material having the surface roughness (Ra) of 0.2 to 0.4 so that the bacterial film is not formed.

### BRIEF DESCRIPTION OF THE DRAWINGS

FIG. 1a is a photograph (expansion in left and right directions) of a front surface of a mouthpiece according to an embodiment of the present invention.

FIG. 1B is a photograph of a rear surface of the mouthpiece according to an embodiment of the present invention.

FIG. 1c is a photograph of a left surface of the mouthpiece according to an embodiment of the present invention.

FIG. 1d is a photograph of a right surface of the mouthpiece according to an embodiment of the present invention.

FIG. 1e is a photograph of a top surface of the mouthpiece according to an embodiment of the present invention.

FIG. 1f is a photograph of a bottom surface of the mouthpiece according to an embodiment of the present invention.

FIG. 1g is a photograph of the bottom surface of the mouthpiece according to an embodiment of the present invention, showing a state in which the mouthpiece is coupled to a connecting wire.

FIG. 2a is a photograph (expansion in a forward direction) of a top surface of a mouthpiece according to another embodiment of the present invention.

FIG. 2b is a photograph of a bottom surface of the mouthpiece according to another embodiment of the present invention.

FIG. 2c is a photograph of the bottom surface of the mouthpiece according to another embodiment of the present invention, showing a state in which segment pieces are spaced apart from each other.

FIG. 3a is a photograph (expansion in the left and right directions) of a bottom surface of a mouthpiece according to yet another embodiment of the present invention, showing a state in which the mouthpiece is mounted to a maxillary palate in an oral cavity.

FIG. 3b is a photograph (expansion in the forward direction) of the bottom surface of the mouthpiece according to yet another embodiment of the present invention, showing a boundary line of segment pieces in the state in which the mouthpiece is mounted to the maxillary palate in the oral cavity.

FIG. 4 is a photograph showing an inflammation state of a skin of a palate after the mouthpiece according to yet another embodiment of the present invention is worn. (An amount of a downward flown soft tissue and hard tissue of the palate is relatively far less than that of FIG. 9b, and the inflammation state is far less than that of FIG. 9b. FIG. 4 shows pink gums, and FIG. 9b shows a reddened state.)

FIG. 5a is a photograph of the front surface of the mouthpiece according to an embodiment of the present invention, showing a state in which the mouthpiece is connected to the connecting wire.

FIG. 5b is a photograph of the rear surface of the mouthpiece according to an embodiment of the present invention, showing the state in which the mouthpiece is connected to the connecting wire.

FIG. 5c is a photograph of the top surface of the mouthpiece according to an embodiment of the present invention, showing the state in which the mouthpiece is connected to the connecting wire.

FIG. 5d is a photograph of the bottom surface of the mouthpiece according to an embodiment of the present invention, showing the state in which the mouthpiece is connected to the connecting wire.

FIG. 6a is a schematic side cross-sectional view of a skeleton, showing action between a head gear and the mouthpiece according to an embodiment of the present invention.

FIG. 6b is a photograph of a side cross-sectional view of a skull, showing the action between the head gear and the mouthpiece according to an embodiment of the present invention.

FIG. 6c is a bottom view of a maxillary bone, showing the action (forward expansion+skull rotation) between the head gear and the mouthpiece according to an embodiment of the present invention.

FIG. 6d is a front cross-sectional view of the maxillarybone, showing an action (expansion in the left and right directions) in two directions caused by two screws of the head gear and the mouthpiece according to an embodiment of the present invention.

FIG. 7a is a photograph of the side surface of the mouthpiece including a piezoelectric generator according to yet another embodiment of the present invention.

FIG. 7b is a photograph of the top surface of the mouthpiece including the piezoelectric generator according to yet another embodiment of the present invention.

FIG. 7c is a schematic bottom view of the mouthpiece including the piezoelectric generator according to yet another embodiment of the present invention.

FIG. 7d is a schematic view showing an arrangement of a generator, a power storage, a low frequency generator, an infrared/ultraviolet LED of the mouthpiece including the piezoelectric generator according to yet another embodiment of the present invention.

FIG. 8a to 8c are schematic views showing a configuration of the maxillary bone.

FIG. 9a is a photograph of a bottom surface of a mouthpiece of the related art, in which a gap occurs between segment pieces.

FIG. 9b is a photograph showing an inflammation state (an amount of a downward flown soft tissue and hard tissue of the palate is greater than that of FIG. 4, and the inflammation state is seriously reddened.) of the skin of the palate after the mouthpiece of the related art, in which the gap occurs between the segment pieces, is worn.

FIG. 10a is a schematic side cross-sectional view of the skeleton, showing an action of only the mouthpiece of the related art (without a head gear).

FIG. 10b is a photograph showing a side cross-section of the skull, showing the action of only the mouthpiece of the related art (without the head gear).

FIG. 10c is a front cross-sectional view of the maxillary bone, showing an action (expansion from a center in the left and right directions) in one direction caused by one screw of only the mouthpiece of the related art (without the head gear).

FIG. 10d is a front cross-sectional view of the maxillary bone, showing an action (expansion from one side in the left and right directions) in two directions caused by two screws of only the mouthpiece of the related art (without the head gear).

FIG. 11a is a schematic side cross-sectional view of the skeleton, showing an action (forward expansion of the related art) of the mouthpiece of the related art and a RAMPA head gear of the related art.

FIG. 11b is a photograph showing a side cross-section of the skull, showing the action of the mouthpiece of the related art and the RAMPA head gear of the related art. (As an anterior portion of the palate bone protrudes downward due to side effects caused by the device of FIG. 11a, a mandibulae rotates in a rear downward direction, and the skull vertically increases and horizontally decreases.)

### MODE FOR CARRYING OUT THE INVENTION

Preferred embodiments of the inventive concept will now be described in detail with reference to the accompanying drawings. Advantages and features of the present disclosure, and implementation methods thereof will be clarified through following embodiments described with reference to the accompanying drawings. The present invention may, however, be embodied in many different forms and should not be construed as being limited to the embodiments set forth herein. Rather, these embodiments are provided so that this disclosure will be thorough and complete, and will fully convey the concept of the invention to those skilled in the art. Further, the present invention is only defined by scopes of claims. Like reference numerals refer to like elements throughout.

Unless otherwise defined, all terms (including technical and scientific terms) used herein have the same meaning as generally understood by those skilled in the art. Also, unless defined apparently in the description, the terms are not ideally or excessively construed as having formal meaning.

On the other hand, it will be understood that when an element is directly disposed on or connected to another one, further another element can be present therebetween. Also, a module performing a certain function may be realized by dividing one module into a plurality of modules or integrating a plurality of modules each having a function into one module. Also, any electronic function block may be realized by implementing a software or realizing the software into a hardware through an electrical circuit.

<Overall Configuration>

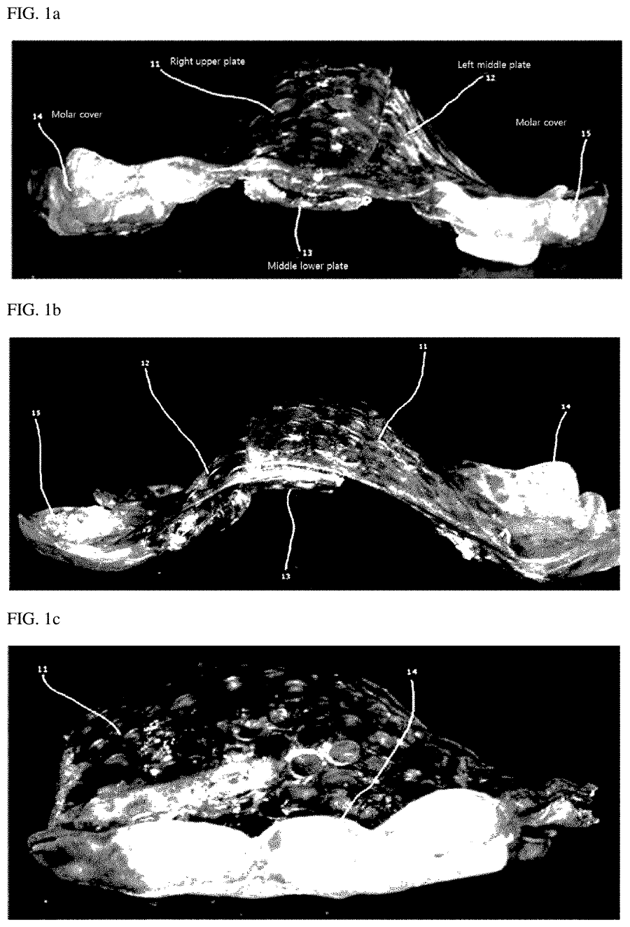

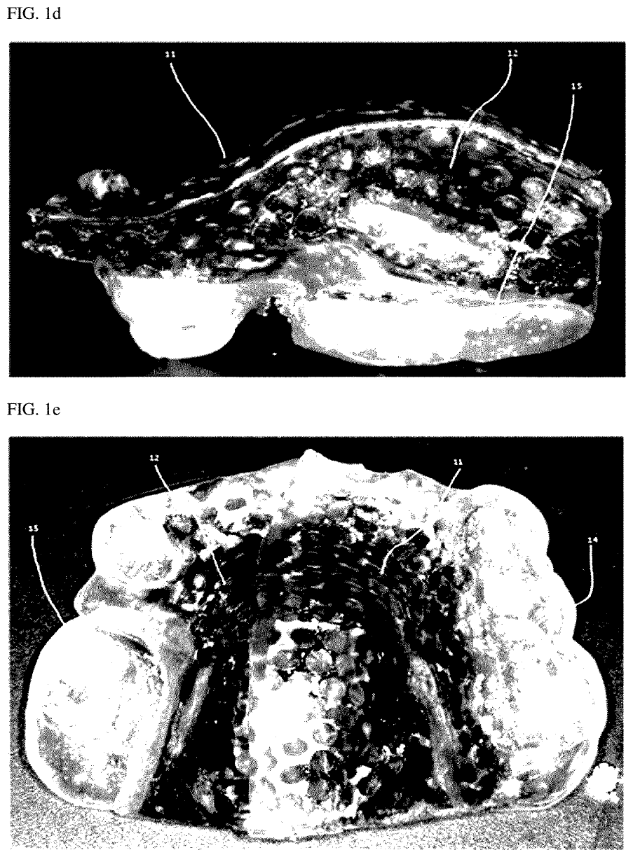

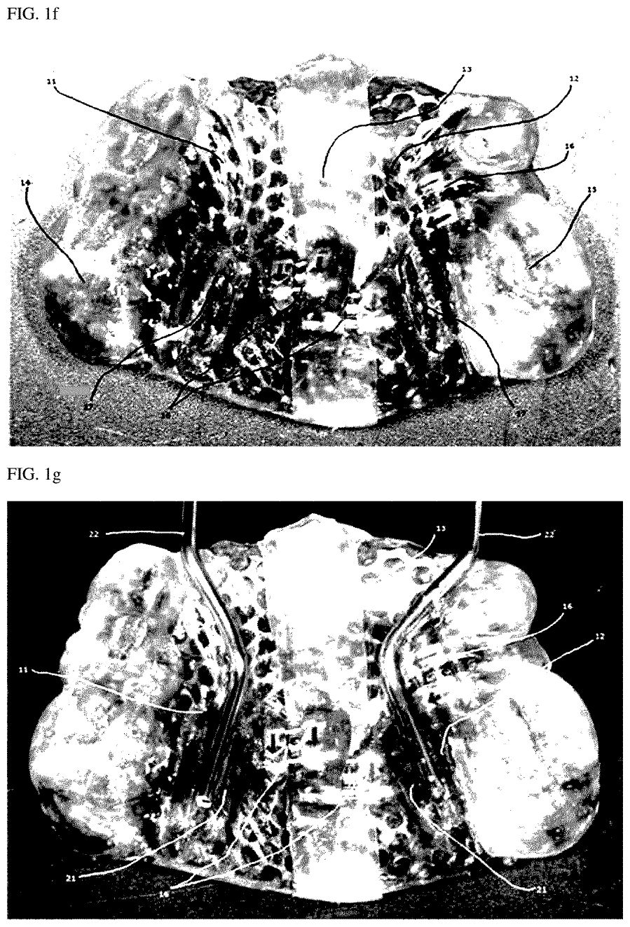

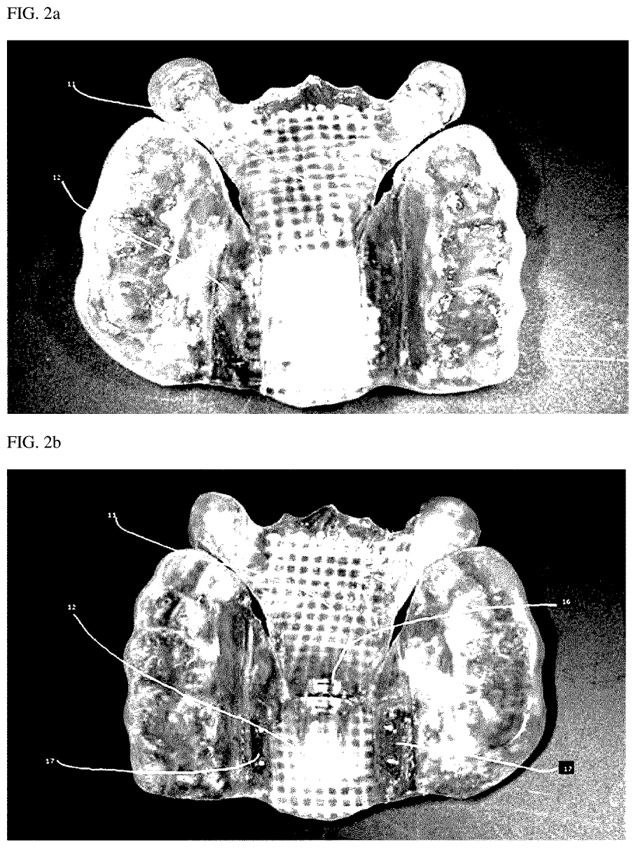

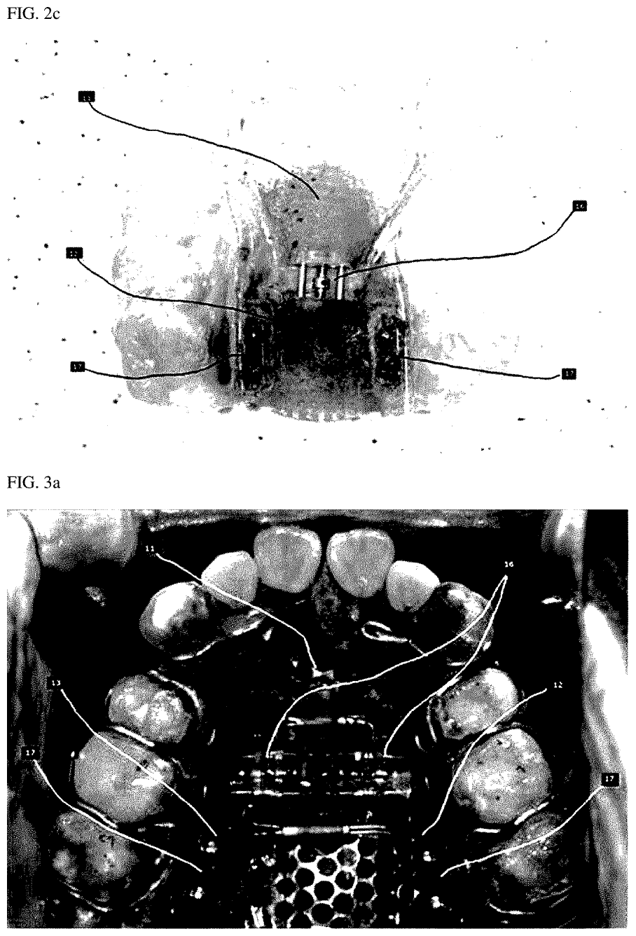

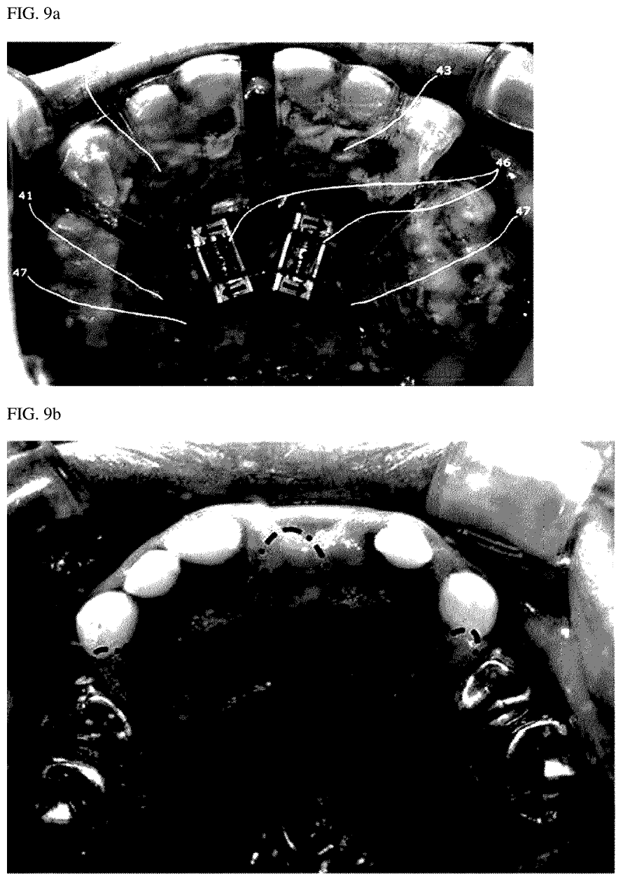

As illustrated in FIGS. 1 and 2, a mouthpiece according to an embodiment of the present invention is mounted in an oral cavity for use in changing a shape of a skull including a maxillary bone and is composed of a plurality of segment pieces 11, 12, and 13. Here, at least some of the segment pieces 11, 12, and 13 are configured to approach or be spaced apart from each other by means of a screw 16. A mouthpiece of the related art illustrated in FIG. 9a may have the above-described configuration and function. A segment piece 41 and a segment piece 42 may approach or be spaced apart from each other by a screw 46, and the segment piece 41 and a segment piece 43 may approach or be spaced apart from each other by another screw 46. Likewise, in the mouthpiece according to an embodiment of the present invention illustrated in FIG. 1, a segment piece 13 and a segment piece 11 may approach or be spaced apart from each other by a screw 16, and the segment piece 13 and a segment piece 12 may approach or be spaced apart from each other by another screw 16. In a mouthpiece according to another embodiment of the present invention illustrated in FIG. 2, a segment piece 12 and a segment piece 11 may approach or be spaced apart from each other by a screw 16.

In the mouthpiece according to an embodiment of the present invention, surfaces of the segment pieces 11, 12, and 13 facing or contacting a skin in the oral cavity, i.e., a skin of a palate, are configured such that the segment pieces 11, 12, and 13 overlap with each other so that a gap is not generated in spite of an approaching or spacing-apart movement of the segment pieces relative 11, 12, and 13 to each other. That is, the mouthpiece according to an embodiment of the present invention is characterized in that the segment pieces 11, 12, and 13 overlap with each other.

Referring to a structure of the mouthpiece of the related art in FIG. 9a, the segment pieces 41, 42, and 43 are not in overlap with each other. Thus, when the segment pieces 41, 42, and 43 are spaced apart from each other by expansion of the mouthpiece caused by the screw, a gap is generated between the segment pieces 41, 42, and 43. The skin of the oral cavity, i.e., the skin (or tissue) of the palate, is exposed through the gap. This exposed tissue of the palate flows downward, and inflammation (a reddened surface outside an oval expressed by a dash-dotted line) is developed on surfaces contacting the mouthpiece device due to contact with foods as in FIG. 9b to cause pain, thereby causing reluctance to wear the mouthpiece.

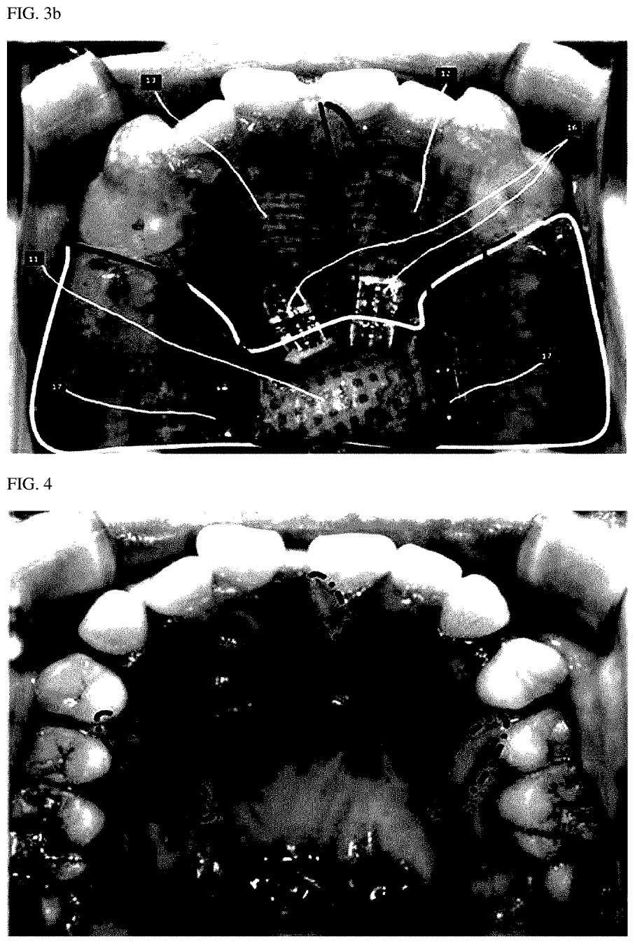

Referring to the structure of the mouthpiece according to an embodiment of the present invention in FIGS. 1 and 2, since the segment pieces 41, 42, and 43 overlap each other as in FIG. 3b, although the segment pieces 41, 42, and 43 are spaced apart from each other when the expansion of the mouthpiece caused by the screw, the gap is not generated between the segment pieces 41, 42, and 43. Thus, since the tissue of the palate is not exposed by using the mouthpiece according to an embodiment of the present invention, downward flow (a surface inside a dash-dotted line) of a soft tissue and a hard tissue is minimized as in FIG. 4, and a risk of inflammation (a surface outside the dash-dotted line) caused by the contact with foods is minimized as in FIG. 4 to prevent pain, thereby remarkably reducing the reluctance to wear the mouthpiece.

<Mouthpiece Canceling Reaction by Headgear>

In the mouthpiece according to an embodiment of the present invention, at least a portion of the segment pieces 11, 12, and 13 may be connected to the head gear mounted outside the oral cavity, and a reaction force vector to an action force vector caused by the screw 16 may be set to be canceled by a support force vector caused by the head gear.

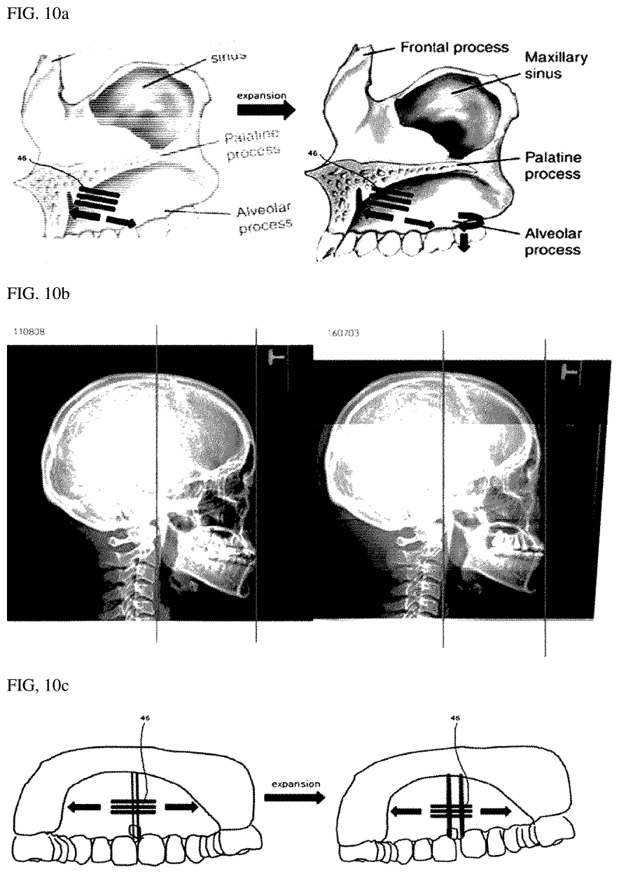

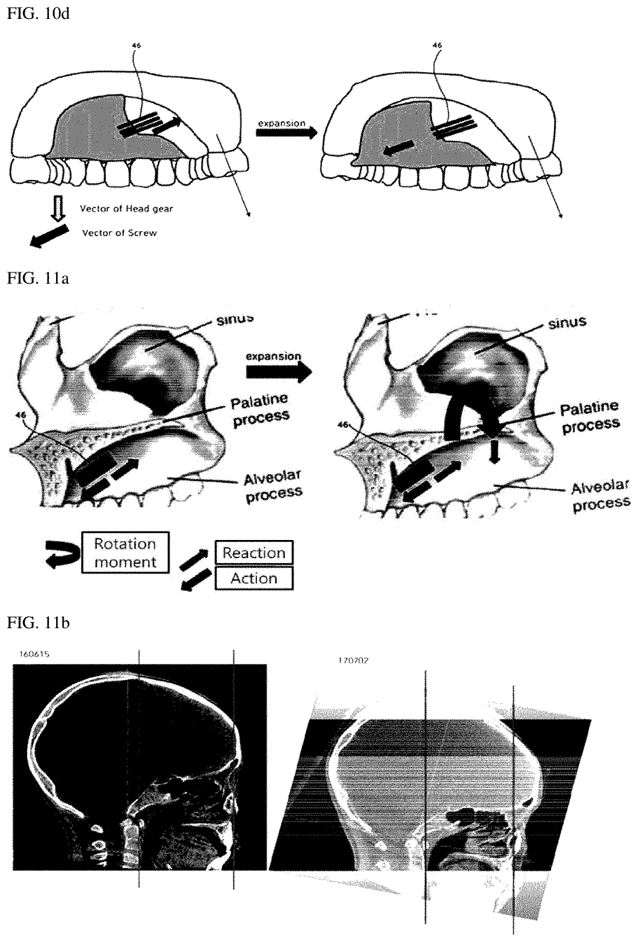

In case of the mouthpiece of the related art, only the mouthpiece is worn in the oral cavity without the head gear. In this case, when the action force vector (red arrow) is applied to an anterior upper portion of the palate by the expansion of the screw 46 as in FIG. 10a, the reaction force vector (blue arrow) acts in a direction opposite thereto. Here, as a posterior contact portion of the device is displaced downward from a mucous membrane in the oral cavity, a posterior portion of the palatine bone is not supported. Accordingly, posterior teeth protrude downward. Here, as a cervical portion of the posterior teeth in contact with a resin phase of the device is used as a center of rotation, a momentum (less in magnitude than a momentum of a support of anterior teeth) by which the palatine bone rotates downward by using a working distance that is a distance between the cervical portion of the posterior teeth and the palatine bone is generated. Also, since the posterior portion is not supported, the anterior portion may not be expanded in a foward direction. Also, the device is lifted from the tissue to cause deformation. However, when a patient has a good posture so that a tongue contacts the palatine bone, the downward displacement of the posterior teeth may be prevented. In this case, skeleton correction of the skull is insufficiently performed as in FIG. 10b.

Here, when the screw acts in one direction by one screw, preferred correction may not be performed as in FIG. 10c, and when the screw acts in two directions by two screws, as the resin portion of the device slips along the cervical portion and moves toward an occlusal surface as in FIG. 10d, a coupling force and a reaction between the mouthpiece device and the palatine surface may not be adjusted.

Also, even when the mouthpiece is coupled to the head gear, adjustment of the action force or resolving of side effects may not be properly performed. For example, when the mouthpiece of the related art is applied to the RAMPA invented by Dr. Mitani and driven by the head gear, the posterior contact portion of the device is displaced downward from the mucous membrane in the oral cavity, and the posterior portion of the palatine bone is not supported. Accordingly, the anterior teeth protrude in a forward and downward direction. When the device is expanded, the cervical portion of the anterior teeth that is a push point is used as a center of rotation, a distance from the point to the skull is a working distance, and a momentum by which the posterior portion of the palatine bone rotates downward is generated. This is a big momentum because of a long arm length. Also, a forward expansion of the anterior portion is insufficient because the posterior portion is not supported. Also, the device is lifted from the tissue to cause deformation. Also, as a mandibulae rotates in a rear downward direction, a face is increased in length. In this case, in the skull as in FIG. 11b, as a forward and backward distance from an anterior cervical vertebrae to mandibular anterior teeth greatly decreases, the maxillary anterior teeth are displaced downward (until cervical vertebrae 3^{rd}). The correction may not be properly performed, e.g., a narrowed respiratory tract is observed.

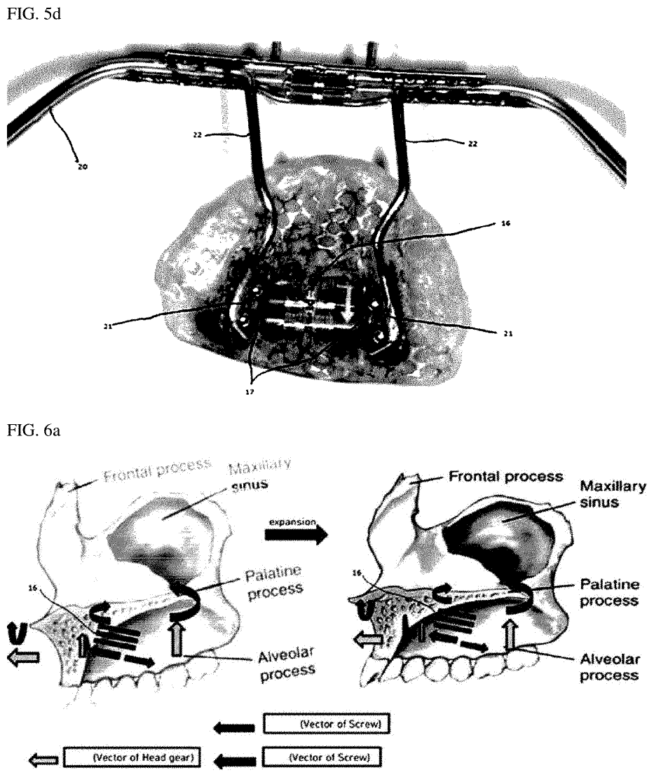

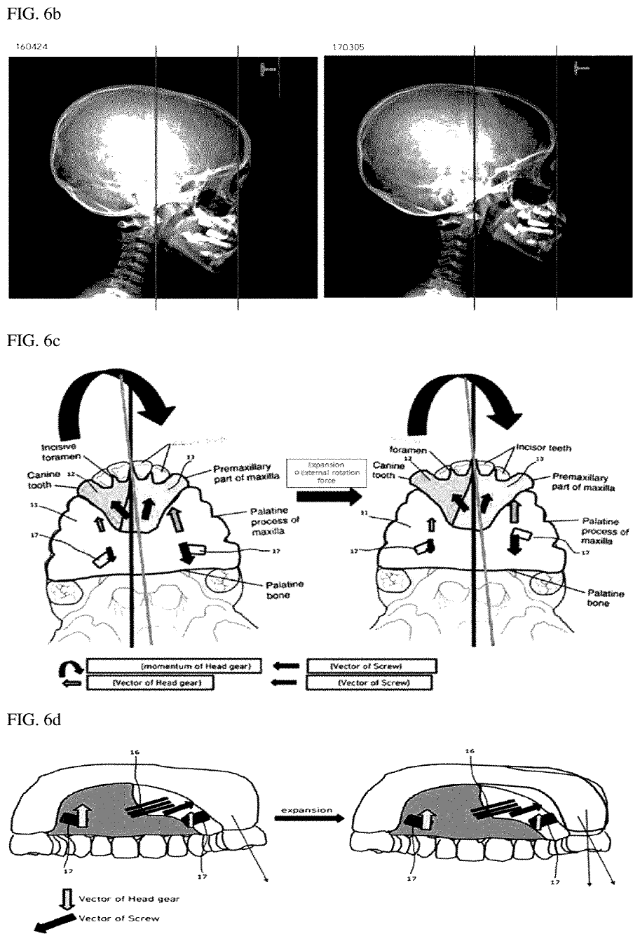

On the contrary, in the mouthpiece according to an embodiment of the present invention, at least a portion of the segment pieces 11, 12, and 13 are connected to the head gear mounted outside the oral cavity. This head gear is a device mounted to a head portion. As illustrated in FIG. 6a, a reaction force vector (blue arrow) to an action force vector (red arrow) caused by the screw 16 may be set to be canceled by a support force vector (green arrow) caused by the head gear. Since the reaction force vector is canceled by the support force vector through the above-described configuration, only the action force vector is remained. Thus, since the action force that is pre-designed for treatment is properly applied to the maxillary bone, treatment effects may be surely controlled. FIG. 6b shows pictures of the skull showing the effects of the correction.

Here, when a correction process of the palate viewed from the bottom is reviewed, as illustrated in a left drawing of FIG. 6c (bottom view), the correction process sets a correction purpose of applying the action force vector (red arrow) to the maxillary bone (blue line) twisted to a right side (left side in the drawing) from a center (red line). Also, with respect to the segment piece 11, the segment piece 12 moves forward obliquely in a right forward direction (left upper direction in the drawing), and the segment piece 13 moves forward obliquely in a left forward direction (right upper direction in the drawing). That is, although a force for correction is applied in the forward direction, forces applied to the left side and the right side are different.

In this case, the segment piece 11 supported by the head gear disposed outside the oral cavity through a holder 17 generates a force to cancel the reaction force vector (blue arrow) generated in a direction opposite to the action force vector (red arrow), i.e., the support vector (green arrow). The support vectors in the left and right sides are different. In case of the exemplary case, the left support vector (right side of the drawing) is greater than the right support vector (left side of the drawing). Accordingly, as a momentum by which the maxillary bone rotates from the right side to the left side (from the left side to the right side in the drawing) is generated, a center of the maxillary bone is coincided to the center (red line) as in a right drawing of FIG. 6c.

<Connecting Wire Coupling Method>

At least a portion of the segment pieces 11, 12, and 13 may be connected to a connecting wire 20, and the connecting wire 20 may be connected to the head gear mounted outside the oral cavity. That is, the connecting wire 20 is disposed between the mouthpiece and the head gear instead of directly mounting the mouthpiece to the head gear.

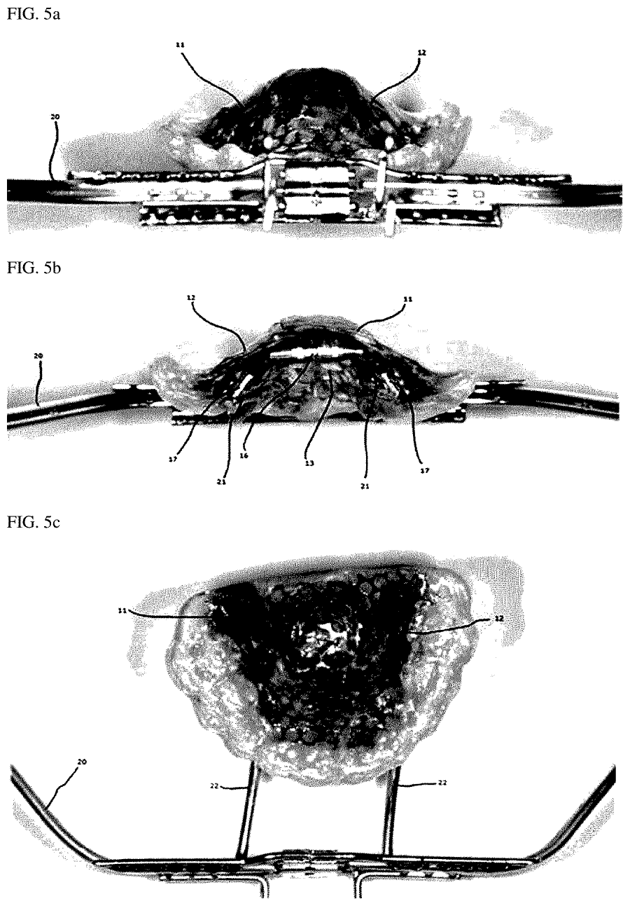

In this case, as illustrated in FIG. 5, a blind hole or a through-hole may be formed in the holder 17 disposed on a bottom surface of each of the segment pieces 11 and 12 connected to the connecting wire 20, and a projection (convex shape) that is detachably coupled to the blind hole or the through-hole is formed on a latch 21 of the connecting wire 20.

For example, in embodiments of FIGS. 2, 3, and 5, a projection is formed on the holder 17 of the segment piece 12, and a hole is defined in latch 21 of an arm 22 of the connecting wire 20 in contrary to the embodiment of FIG. 1. In this case, when the latch 21 is coupled to the holder 17, as a protruding shape of the projection of the holder 17 protruding through the hole of the latch 21 of the arm 22 is formed on the bottom surface of the segment piece 12 in addition to the arm 22 of the connecting wire 20, there is a problem in that an active space of the tongue is greatly restricted.

On the contrary, in the embodiment of FIG. 1, as a hole is defined in the holder 17 of the segment piece 13, and the projection is formed on the latch 21 of the connecting wire 20, when the mouthpiece is mounted in the oral cavity, the projection of the latch 21 of the arm 22 of the connecting wire 20 is inserted to the hole of the holder 17 of the segment piece 13. Thus, there is an advantage in that the active space of the tongue is largely secured because a protruding shape of only the arm 22 of the connecting wire 20 is formed on the bottom surface of the segment piece 13.

<Electrical System>

At least one of the segment pieces 11, 12, and 13 may include a generator 31a for converting a masticatory force in the oral cavity into electrical energy by a piezoelectric phenomenon or a power storage 31b for storing the electrical energy.

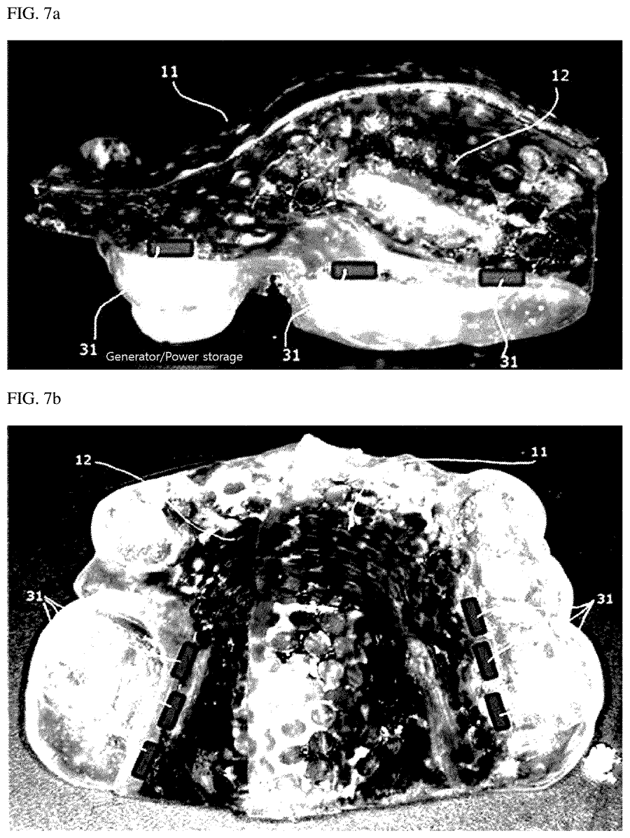

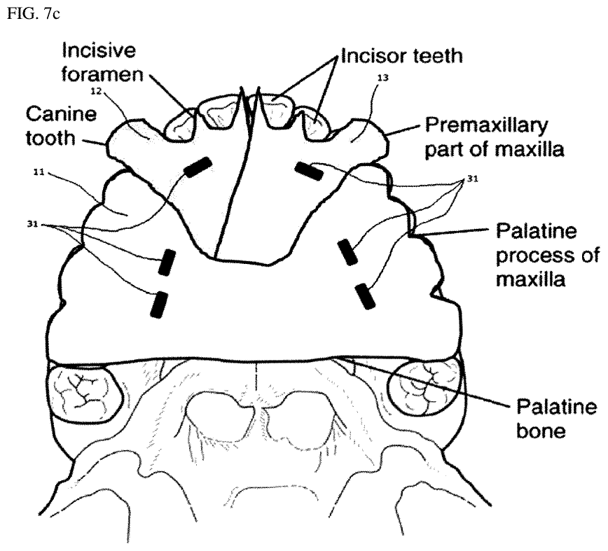

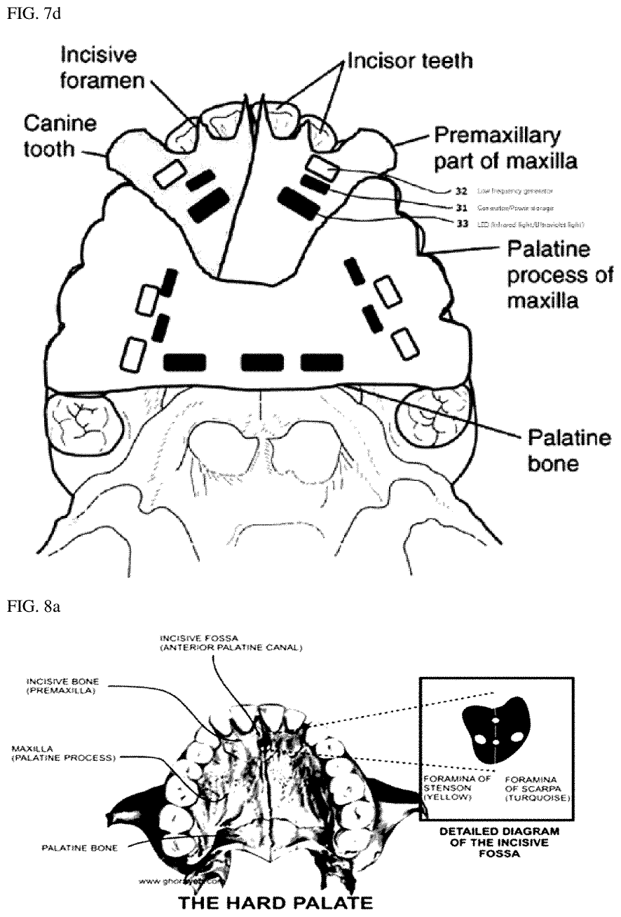

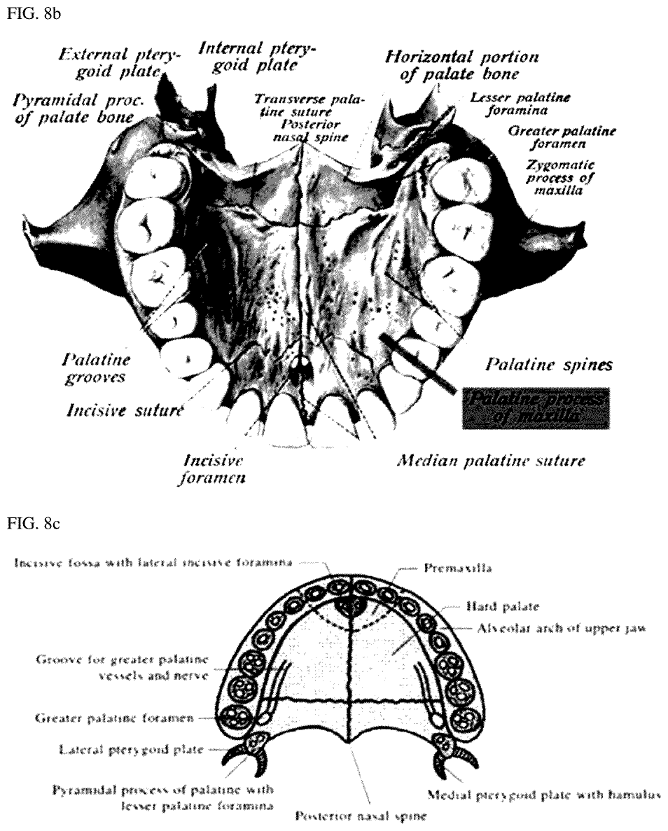

The segment piece including the generator 31a or the power storage 31b may be, e.g., a segment piece including a molar tooth cover 14 and 15 coupled to a molar tooth or a segment piece 11 and 12 in case of FIG. 1, which is illustrated in a side view of FIG. 7a, a top view of FIG. 7b, and a bottom view of FIG. 7c. Here, an embedded position of the generator 31a or the power storage 31b may be positioned to the cervical portion of the tooth because surfaces contacting each other while masticating foods may be deleted when occlusal adjustment is required.

For example, the generator 31a may include a piezoelectric power generation element, and the power storage 31b may include a condenser or a battery. For example, a MEMS piezoelectric sensor is embedded in a road surface of a bridge and converts a pressure caused by a vibration generated while vehicles pass into electricity to be used as electric power for a street lamp of a bridge and CCTV. The generator or the power storage may effectively convert mechanical energy caused by the masticatory movement into electrical energy to be directly used or used after being stored.

The converted electrical energy may drive at least one of a low frequency generator, an infrared LED, an ultraviolet LED, a screw drive motor, a wireless communication module with an external terminal, and a screw actuation amount sensor. For example, the above-described actuators, modules, or sensors may be disposed as in FIG. 7d.

The low frequency generator may be configured to receive the electrical energy from the generator or the power storage and apply a low frequency that helps, e.g., muscle relaxation and blood circulation to the skin of the palate. The low frequency generator transmits an electrical stimulation having a frequency less than 250 Hz to bioelectricity that minutely flows through human bodies to repeat contraction and relaxation of a muscle.

The infrared LED may be configured to apply infrared light having a frequency that helps bone regeneration to the palate. Far-infrared radiation (FIR) may be realized by being coated in a film shape, and the far-infrared radiation is radiated at a body temperature. This has effects such as skin regeneration promotion, muscle relaxation, and pain reduction. Also, power may be supplied through graphene.

The ultraviolet LED may be configured to apply ultraviolet light having a frequency of sterilizing harmful bacteria in the oral cavity to the palate. The UV sterilization may sterilize 97% of harmful microorganisms with a wavelength of 282 nm during 2.5 hours. This may be effective in reducing the harmful bacteria in the oral cavity. Here, the LED power is automatically turned-on one time a day, and a time is adjusted to prevent symbiotic bacteria from dying.

The screw drive motor may be driven by a manual or automatic control signal and controlled to rotate the screw 16 in a forward direction or a reverse direction. For example, the control signal may act such that the control signal is sent from a smartphone of a user, received through the wireless module connected to the screw 16, and then transmitted to a driving circuit of the screw drive motor.

For example, the wireless communication module with the external terminal may wirelessly communicate with the smartphone of the user, e.g., the Bluetooth method. All information including a device used in the oral cavity (all sorts of general devices used in the oral cavity such as orthopedic devices, occlusal devices, splints, and sleep apnea prevention devices), a mounting time of the device, a force applied to the device, a tensile force, and a torsional force is stored through wireless communication, and then data such as treatment information may be transmitted to a terminal, e.g., a coupled mobile phone of a patient, every six hours. For example, the wireless communication module may be configured to further transmit information from the terminal to a sub-server. Also, a doctor may receive data from a server to check and evaluate whether the device for the patient is mounted as planned. The data may be used for device development.

The screw actuation sensor detects information of a rotation amount and direction of the screw. From the information, a distance between the segment pieces may be known.

In case of a sensor, for example, a piezoelectric power generator may be mounted to each component of the device to individually measure a force applied to the component. Here, a serial number may be assigned to each component.

Also, the mouthpiece according to the present invention is characterized by being made of a material having surface roughness (Ra) of 0.2 to 0.4 so that a bacterial film is not formed on an entire surface of the mouthpiece or a surface of a portion of each of the segment pieces.

When the mouthpiece is manufactured by a typical method of pouring general correction resin into a frame and then curing the correction resin, the surface roughness (Ra) may exceed 0.4. Here, as various shapes and sizes of concave portions such as a crater on the moon are generated on the surface of the mouthpiece, and a porous inner space generated when the resin is evaporated to the outside during a curing process is formed, various foods in a solid, semi-solid, fluid, or liquid state may be inserted thereto.

In consideration of the above-described feature, the mouthpiece according to the present invention may be made of a material having the surface roughness (Ra) of 0.2 to 0.4. When the surface roughness (Ra) is less than 0.2, a high-priced material is necessary to be used to remarkably increase a manufacturing cost and also extremely increase a cost or a time for surface finish treatment. When the surface roughness (Ra) is greater than 0.4, remained foods cause bacteria propagation, and an odor generation rate is extremely increased within four hours to exceed a reference.

By managing the surface roughness as described above, the number of structures such as porous portions, concave portions, or convex portions used for bacteria propagation is decreased, or a remaining possibility of foods is minimized Thus, a cause of odor generation may be prevented in advance, and skin flare and stinging caused by bacterial infection may be prevented to exhibit advantageous sanitary effects

The description of the present invention is intended to be illustrative, and those with ordinary skill in the technical field of the present invention will be understood that the present invention can be carried out in other specific forms without changing the technical idea or essential features. Thus, the above-disclosed embodiments are to be considered illustrative and not restrictive.

### INDUSTRIAL APPLICABILITY

The present invention may be used in the mouthpiece industry.

### DESCRIPTION OF REFERENCE NUMERAL

11, 12, 13, 41, 42, 43: Segment piece
14, 15: Tooth cover
16, 46: Screw
17, 47: Holder
20: Connecting wire
21: Latch
22: Connecting wire arm
31: Generator/Power storage
32: Low frequency generator
33: LED (Infrared light/Ultraviolet light)

1. A mouthpiece comprising:
a plurality of segment pieces mounted in an oral cavity to press an inner wall of the oral cavity or a tooth; and
a plurality of screws configured to connect at least a portion of the plurality of segment pieces and adjust a distance between the connected segment pieces.

2. The mouthpiece of claim 1, further comprising a low frequency generator disposed on at least one of the plurality of segment pieces to repeat contraction and relaxation of a muscle in the oral cavity through electrical stimulation.

3. The mouthpiece of claim 1, further comprising an infrared LED disposed on at least one of the plurality of segment pieces to emit infrared light and having at least one effect of bone regeneration in the oral cavity, skin regeneration in the oral cavity, muscle relaxation in the oral cavity, and oral pain reduction.

4. The mouthpiece of claim 1, further comprising an ultraviolet LED disposed on at least one of the plurality of segment pieces to emit infrared light having a frequency of sterilizing harmful bacteria in the oral cavity,
wherein the ultraviolet LED is automatically turned on and off as many as a predetermined number of times during a reference time.

5. The mouthpiece of claim 1, further comprising a screw drive motor disposed on at least a portion of the plurality of screws to rotate the plurality of screws in a forward direction or a reverse direction so that a distance between the plurality of segment pieces is adjusted.

6. The mouthpiece of claim 5, further comprising a wireless communication module connected with the screw drive motor,
wherein the screw drive motor is controlled by an external terminal through the wireless communication module.

7. The mouthpiece of claim 6, further comprising a screw actuation amount sensor configured to measure a rotation direction and a rotation amount of the screw drive motor,
wherein information of the rotation direction and the rotation amount measured by the screw actuation amount sensor is transmitted to the external terminal through the wireless communication module, and the external terminal calculates a spaced distance between the plurality of segment pieces based on the information of the rotation direction and the rotation amount.

8. The mouthpiece of claim 1, further comprising:
a sensor disposed on the plurality of segment pieces to measure at least one of a force applied to the segment piece and a displacement of the segment piece; and
a wireless communication module configured to communicate wirelessly with the sensor and an external terminal,
wherein information measured by the sensor is transmitted to the external terminal.

9. The mouthpiece of claim 8, wherein the sensor is a piezoelectric sensor configured to convert the force applied to the segment piece into electrical energy, and
the mouthpiece further comprises a power storage configured to store the electrical energy converted by the piezoelectric sensor.

10. An oral cavity state monitoring system using the mouthpiece of claim 8, comprising:
an external terminal configured to transceive information with the wireless communication module; and
a server configured to transceive information with the external terminal,
wherein the external terminal transmits the information received from the wireless communication module to the server at a predetermined time interval.
